(19) United States
(12) Patent Application Publication
(10) Pub. No.: US 2016/0135486 A1
(43) Pub. Date: May 19, 2016

(54) Method for Producing a Food Product

(71) Applicant: NOVOZYMES A/S, Bagsvaerd (DK)

(72) Inventors: Hanne Vang Hendriksen, Bagsvaerd (DK); Katja Puder, Bagsvaerd (DK); Martin Johannes Baumann, Bagsvaerd (DK); Gitte Budolfsen Lynglev, Bagsvaerd (DK); Allan Noergaard, Lyngby (DK); Martin Simon Borchert, Bagsvaerd (DK)

(73) Assignee: Novozymes A/S, Bagsvaerd (DK)

(21) Appl. No.: 14/897,485

(22) PCT Filed: Jun. 23, 2014
(86) PCT No.: PCT/EP2014/063112

§ 371 Date: Dec. 10, 2015

(30) Foreign Application Priority Data
Jun. 24, 2013 (EP) 13173385.9

(51) Int. Cl. A23F 5/04; A23F 5/16; C12N 9/82; C12N 1/21; A23L 1/216; A23L 1/217

(52) U.S. Cl. CPC A23L 1/0153; C12N 9/82; A23F 5/163; A23L 1/164; A23L 1/217; A23L 1/2161; A23L 1/2165; A23L 1/2163; C12Y 305/01001; A23V 2002/00

(57) ABSTRACT

The present invention relates to a method for producing a heat-treated food product from a food material which has been contacted with an asparaginase.

### REFERENCE TO SEQUENCE LISTING

This application contains a Sequence Listing in computer readable form. The computer readable form is incorporated herein by reference.

### FIELD OF THE INVENTION

The present invention relates to a method for producing a heat-treated food product from a food material which has been contacted with an asparaginase.

### BACKGROUND OF THE INVENTION

It is well known that acrylamide formation in heated food products may be reduced by a treatment reducing the amount of asparagine in the food materials, such as by subjecting the food materials to the action of the enzyme asparaginase (see e.g. WO2004/026042).

WO2004/032648 and WO2004/030468 disclose asparaginases from Aspergillus oryzae and Aspergillus niger, respectively, and their use in food production. WO2008/151807 discloses a hyper-thermostable asparaginase from Pyrococcus furiosus and its use in food production.

These asparaginases are all useful in industrial food manufacturing in different processes. However, to fit into the production line of an industrial food product, treatment with asparaginase should preferentially take place during an existing step in the production process. Therefore, the availability of food manufacturing processes using different asparaginases having different properties, such as different thermostability, different thermoactivity, different pH optimum, different pH stability, different inhibitors of activity, different dosage requirements, etc., is desirable.

### BRIEF DESCRIPTION OF DRAWINGS

FIG. 1 shows DSC thermograms at pH 5 for Thermococcus kodakaraensis asparaginase (dotted curve) and Pyrococcus furiosus asparaginase (solid curve).

FIG. 2 shows DSC thermograms at pH 7 for Thermococcus kodakaraensis asparaginase (dotted curve) and Pyrococcus furiosus asparaginase (solid curve).

### SUMMARY OF THE INVENTION

The present inventors have found that an asparaginase obtained from Thermococcus kodakaraensis (SWISSPROT:Q5JIW4) having the amino acid sequence of SEQ ID NO: 10 is useful in the production of heat-treated food products. The inventors have found that the enzyme is thermostable. Further, the inventors found that the asparaginase of the present invention is clearly more efficient than the asparaginase from P. furiosus in some application trials when comparing on an enzyme protein basis.

The invention therefore provides a method for producing a heat-treated food product comprising:

(a) contacting of a food material with an asparaginase which (i) has at least 60% sequence identity to the mature polypeptide of SEQ ID NO: 10, (ii) is encoded by a polynucleotide having at least 60% sequence identity to SEQ ID NO: 9, or (iii) is a variant of the mature polypeptide of SEQ ID NO: 10 comprising a substitution, deletion, and/or insertion at one or more positions; and

(b) heat-treating the asparaginase treated food material to obtain the heat-treated food product.

The invention also provides an isolated polynucleotide having at least 60% sequence identity to SEQ ID NO: 9, which encodes an asparaginase; as well as nucleic acid constructs, recombinant expression vectors, recombinant host cells comprising the polynucleotides, and methods of producing an asparaginase.

### DETAILED DESCRIPTION OF THE INVENTION

In the context of the present invention, the term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C terminal and/or N terminal amino acid) expressed by the same polynucleotide.

Based on N-terminal sequencing and mass spectrometry (MS) analysis, the mature polypeptide of SEQ ID NO: 10 is amino acids 1 to 328 of SEQ ID NO: 10.

The invention provides a method for producing a heat-treated food product. A food product according to the invention is any nutritious substance that people eat or drink. For the avoidance of any possible doubt, this includes roasted coffee beans.

The food product is obtained from a food material which is to be contacted with an asparaginase for an appropriate time interval allowing the asparaginase to exert its action. The time interval for the contacting depends on various factors, such as the production process, the food material, the asparaginase concentration, etc. The skilled person will readily be able to determine the contacting time.

In a preferred embodiment, the contacting in step (a) is for at least 2 minutes, e.g., at least 3 minutes. In some embodiments, the contacting in step (a) is for at least 5 minutes, e.g., at least 10 minutes.

In another preferred embodiment, the contacting in step (a) is for between 2 minutes and 2 hours, preferably for between 3 minutes and 1 hour.

The contacting with the asparaginase constitutes the asparaginase treatment.

After the contacting with the asparaginase in step (a), the asparaginase treated food material is subjected to a heat treatment to obtain the heat-treated food product. The heat treatment may involve, e.g., frying, baking, toasting or roasting.

The contacting with the asparaginase may be performed by the food material being immersed into or sprayed with an asparaginase solution. This is the case for, e.g., food products prepared from cuts of vegetables, such as French fries or sliced potato chips. This may also be the case for coffee-based food products, e.g., roasted coffee beans or coffee prepared by extraction therefrom, where the green coffee beans may be soaked in or sprayed with an asparaginase solution. Alternatively, the contacting with the asparaginase may be performed by mixing the asparaginase into the food material. This may be the case for, e.g., dough products (bread, crackers, corn chips, etc.), breakfast cereals, mashed potatoes, etc. However, food materials of the latter type may also be contacted with asparaginase by the food material, e.g., pieces of dough, being immersed into or sprayed with an asparaginase solution.

The contacting with the asparaginase may be performed, e.g., by the food material being dipped into or sprayed with an asparaginase solution followed by resting or incubation of the food material under conditions where the enzyme is active, e.g., by drying of the food material prior to frying or baking. In that case, the contacting with the asparaginase includes both the dipping/spraying and the resting/incubation/drying. I.e., the food material is contacted with the asparaginase for as long as the food material is in contact with active enzyme. The heat-treatment of step (b) will normally inactivate the asparaginase. I.e., the food material may be contacted with the asparaginase from the initial contact (such as by dipping the food material into or spraying the food material with asparaginase solution or by blending asparaginase into the food material) until the enzyme is inactivated, e.g., by the heat treatment of step (b).

In a preferred embodiment, the asparaginase is added to the food material at a temperature of at least 60° C. In another preferred embodiment, the asparaginase is added to the food material at a temperature of at least 80° C. In another preferred embodiment, the asparaginase is added to the food material at a temperature of 60-110° C. In another preferred embodiment, the asparaginase is added to the food material at a temperature of 80-105° C.

The food material which is to be contacted with the asparaginase according to the method of the invention may be any raw material which is to be included in the food product, or it may be any intermediate form of the food product which occurs during the production process prior to the heating step to obtain the heat-treated food product. It may be any individual raw material used and/or any mixture thereof and/or any mixture thereof also including additives and/or processing aids, and/or any subsequently processed form thereof.

The food product may be made from at least one raw material that is of plant origin, for example a vegetable tuber or root, such as but not limited to the group consisting of potato, carrot, beet, parsnip, parsley root, celery root, sweet potato, yams, yam bean, Jerusalem artichoke, radish, turnip, chicory root and cassava; cereal, such as but not limited to the group consisting of wheat, rice, corn, maize, rye, barley, buckwheat, sorghum, oats and ragi; coffee; cocoa; chicory; olives; prunes or raisins. Also food products made from more than one raw material are included in the scope of this invention, for example food products comprising both wheat (e.g., in the form of wheat flour) and potato.

Raw materials as cited above are known to contain substantial amounts of asparagine which is involved in the formation of acrylamide during the heating step of the production process. Alternatively, the asparagine may originate from other sources than the raw materials, e.g., from protein hydrolysates, such as yeast extracts, soy hydrolysate, casein hydrolysate or the like, which are used as an additive in the food production process.

The asparaginase is to be added to the food material in an amount that is effective in reducing the level of asparagine present in the food material. This will result in less acrylamide being formed in the heating step which is to take place after the enzyme treatment. Such methods are disclosed, e.g., in WO04/026043. The methods disclosed in WO04/026043 and all preferences disclosed are incorporated by reference.

After the contacting with the asparaginase, the asparaginase treated food material is subjected to a heat treatment. The heat treatment is a part of the method for producing a food product from the food material (i.e., the raw material or an intermediate form of the food product). In a conventional method, i.e., a method without asparaginase treatment, more acrylamide would be formed during the heat treatment as compared to the method of the invention where at least some of the asparagine of the food material is hydrolysed by the asparaginase.

Preferred heating steps are those at which at least a part of an intermediate form of the food product, e.g., the surface of the food product, is exposed to temperatures at which the formation of acrylamide is promoted, e.g. 110° C. or higher, or 120° C. or higher. The heating step in the method according to the invention may be carried out in ovens, for instance at a temperature of 180-250° C., such as for the baking of bread and other bakery products, or in oil such as the frying of potato chips or French fries, for example at 160-195° C. Or it may be carried out by toasting or roasting, such as by toasting of breakfast cereals or by roasting of coffee beans. In a preferred embodiment heat-treatment means heating to at least 110° C., preferably at least 120° C., for at least 1 minute. In another preferred embodiment heat-treatment means heating to 110-350° C., preferably 120-300° C., for 1-60 minutes.

In a preferred embodiment, the acrylamide content of the heat-treated food product is reduced by at least 25%, preferably at least 30%, at least 35%, at least 40%, at least 45% or at least 50%, compared to the acrylamide content of a heat-treated food product produced by a similar method without the addition of asparaginase.

In one embodiment of the invention, the heat-treated food product is a cereal-based dough product. It may be a baked cereal-based dough product, such as, e.g., bread, crisp bread, crackers, biscuits, pastry, cake, pretzels, bagels, Dutch honey cake, cookies, gingerbread, ginger cake or baked dough-based chips. Or it may be a fried cereal-based dough product, such as, e.g., corn chips, tortilla chips or taco shells. Cereals may be defined as grasses which are cultivated for the edible components of their grains. In one embodiment, the cereal-based dough product comprises at least one of wheat, rice, corn, maize, rye, barley, buckwheat, sorghum and/or oats. A cereal-based dough may be defined as any mixture comprising at least one cereal-based ingredient and a consumable liquid, with a consistency suitable to be formed into a food product having a definite shape, either by forming the dough directly into such shape or by pouring the dough into a form prior to baking. The food material which is to be contacted with asparaginase may be one or more cereal-based ingredients (for example wheat flour or processed corn), the initial mixture thereof with other ingredients, such as for example water, oil, salt, yeast and/or bread improving compositions, the mixed dough or the corn masa, the kneaded dough, the leavened dough or the partially baked or fried dough or corn masa. The food material may be contacted with asparaginase at a concentration of 0.01-20 mg enzyme protein per kg dry matter, preferably 0.05-10 mg enzyme protein per kg dry matter, more preferably 0.1-5 mg enzyme protein per kg dry matter.

In another embodiment of the invention, the heat-treated food product is a breakfast cereal. The food material which is to be contacted with asparaginase comprises whole or processed cereal kernels or grains, e.g., whole wheat flour, wheat flour, oat flour, corn flour, rice flour, rye flour, wheat kernels, oat kernels or oat flakes. The contacting with asparaginase may be performed by mixing the asparaginase into the food material. The food material may be contacted with asparaginase at a concentration of 0.01-20 mg enzyme protein per kg dry matter, preferably 0.05-10 mg enzyme protein per kg dry matter, more preferably 0.1-5 mg enzyme protein per kg dry matter. The asparaginase may be added to the food material at a temperature of at least 60° C., preferably at least 80° C. In a preferred embodiment, the asparaginase may be added to the food material at a temperature of 60-110° C., preferably 80-105° C. The heat-treatment of the asparaginase treated food material may be performed by toasting. Toasting may be defined as heating by exposure to radiant heat.

In another embodiment of the invention, the heat-treated food product is a potato-based food product, where the food material to be contacted with asparaginase is mashed potato, a potato-based dough or a suspension of a dehydrated potato product, such as potato flakes or granules. Such food product may be, e.g., dough-based potato snacks, fabricated potato products or croquettes. The food material may be contacted with asparaginase at a concentration of 0.01-20 mg enzyme protein per kg dry matter, preferably 0.05-10 mg enzyme protein per kg dry matter, more preferably 0.1-5 mg enzyme protein per kg dry matter. The asparaginase may be added to the food material at a temperature of at least 60° C., preferably at least 80° C. In a preferred embodiment, the asparaginase may be added to the food material at a temperature of 60-100° C., preferably 80-100° C., more preferably 90-95° C. The heat-treatment of the asparaginase treated food material may be performed by frying or baking or a combination thereof.

In another embodiment of the invention, the heat-treated food product is a food product made from cuts of potatoes or other root vegetables such as, but not limited to, carrot, beet, parsnip, parsley root, celery root and cassava, which are fried and/or baked. Examples of such food products are French fries, sliced potato chips and sliced chips from root vegetables such as, but not limited to, carrot, beet, parsnip, parsley root and celery root. The food material which is to be contacted with asparaginase may be cuts of potatoes or other root vegetables which have optionally been peeled and/or blanched. The contacting with asparaginase may be performed by the cuts of potatoes or other root vegetables being dipped in, incubated in or sprayed with an asparaginase solution, possibly followed by resting or incubation of the food material under conditions where the enzyme is active. The asparaginase may be added to the food material at a temperature of 60-95° C., such as at a temperature of 65-85° C. I.e., the food material may be dipped in or incubated in an asparaginase solution having a temperature of 60-95° C., such as a temperature of 65-85° C., or the food material having a surface temperature of 60-95° C. may be sprayed with an asparaginase solution. The asparaginase solution may comprise asparaginase at a concentration of 0.5-200 mg enzyme protein (ep)/L, preferably 1-150 mg ep/L, more preferably 2-120 mg ep/L. The heat-treatment of the asparaginase treated food material may be performed by frying or baking or a combination thereof.

In another embodiment of the invention, the heat-treated food product is French fries. The food material which is to be contacted with asparaginase may be cuts of potatoes in the form of wedges or sticks which are of a size and shape suitable for further processing into French fries. In the context of the present invention, French fries is meant to encompass both the final fries ready for consumption and a par-fried pre-product which is to be finally fried or baked before being consumed. Also, French fries is meant to encompass both French fries made from potato sticks and larger French fries made from, e.g., potato wedges. In a preferred embodiment, the cuts of potatoes, such as the potato sticks or wedges, have been blanched before step (a). Blanching may be performed by any method known in the art, e.g., by wet blanching, steam blanching, microwave blanching or infrared blanching. The contacting with asparaginase may be performed by the cuts of potatoes being dipped in, incubated in or sprayed with an asparaginase solution, possibly followed by resting or incubation of the food material under conditions where the enzyme is active. In a preferred embodiment, the blanched cuts of potatoes are dipped in or sprayed with an asparaginase solution followed by drying of the potato cuts under conditions where the asparaginase is active. The asparaginase may be added to the food material at a temperature of 60-95° C., such as at a temperature of 65-75° C. I.e., the food material may be dipped in or incubated in an asparaginase solution having a temperature of 60-95° C., such as a temperature of 65-75° C., or the food material having a surface temperature of 60-95° C. may be sprayed with an asparaginase solution. The asparaginase solution may comprise asparaginase at a concentration of 0.5-200 mg enzyme protein (ep)/L, preferably 1-150 mg ep/L, more preferably 2-120 mg ep/L. The cuts of potatoes, such as the potato sticks or wedges, may further be contacted with (such as by dipping in or spraying with) other substances, e.g., sodium acid pyrophosphate (SAPP) and/or glucose, either before, at the same time or after the contacting with asparaginase. The cuts of potatoes, such as the potato sticks or wedges, may optionally be dried. The drying may take place before, at the same time or after the contacting with the asparaginase. In a preferred embodiment, the drying is performed under conditions where the asparaginase is active. I.e., the contacting with asparaginase is to take place before or during the drying. Drying may be performed in a drier with air circulation where temperature, humidity and/or air flow can be adjusted to the level(s) desired. The heat-treatment of the asparaginase treated food material may be performed by frying or baking or a combination thereof.

In another embodiment of the invention, the heat-treated food product is sliced potato chips. The food material which is to be contacted with asparaginase is sliced potatoes having a size which is suitable for further processing into potato chips. The contacting with asparaginase may be performed by the sliced potatoes being dipped in, incubated in or sprayed with an asparaginase solution, possibly followed by resting or incubation of the food material under conditions where the enzyme is active. The asparaginase may be added to the food material at a temperature of 60-100° C., such as at a temperature of 65-85° C. I.e., the food material may be dipped in or incubated in an asparaginase solution having a temperature of 60-100° C., such as a temperature of 65-85° C., or the food material having a surface temperature of 60-100° C. may be sprayed with an asparaginase solution. In a preferred embodiment, the contacting with asparaginase is performed by the potato slices being blanched for 1-10 minutes at a temperature of 60-100° C., such as at a temperature of 65-85° C., in an aqueous solution comprising the asparaginase. In one embodiment, the sliced potatoes are contacted with asparaginase by means of a dominant bath. In succession, several batches of potato slices are blanched in the asparaginase solution until the soluble materials that extract from the potato slices are in or near equilibrium with the solution. The asparaginase in the dominant bath converts asparagine to aspartic acid, thus creating a driving force for additional asparagine extraction on subsequent additions of batches of potato slices. Extractable materials can equilibrate with the potato slices such that additional soluble potato components do not extract out, with the exception of asparagine, which continues to react and be converted by the asparaginase. The aspartic acid that is formed from the asparagine soaks back into the potatoes and equilibrates. Additional water is added after every batch of sliced potatoes to make up for the solution being removed by the previous batch; this maintains a constant volume of the dominant bath. The asparaginase in the asparaginase solution may be immobilized. The asparaginase solution may comprise asparaginase at a concentration of 0.5-200 mg enzyme protein (ep)/L, preferably 1-150 mg ep/L, more preferably 2-120 mg ep/L. The heat-treatment of the asparaginase treated food material may be performed by frying.

In another embodiment of the invention, the heat-treated food product is a coffee-based food product, e.g., roasted coffee beans or coffee obtained by extraction of the roasted coffee beans, and the food material which is to be contacted with asparaginase is unroasted coffee beans. Unroasted coffee beans may also be referred to as green coffee beans. The green coffee beans may be subjected to a steam treatment before, during or after the contacting with asparaginase. The contacting with asparaginase may be performed by soaking of the green coffee beans in a solution comprising asparaginase. The asparaginase may be added to the green coffee beans at a temperature of at least 60° C., preferably at least 80° C. In a preferred embodiment, the asparaginase may be added to the green coffee beans at a temperature of 60-110° C., preferably 80-105° C. In a preferred embodiment, the contacting with asparaginase is performed by the green coffee beans being soaked in an asparaginase solution at a temperature of at least 60° C. for 10 minutes to 3 hours, preferably for 30 minutes to 2 hours. The coffee beans may be contacted with asparaginase at a concentration of 0.01-20 mg enzyme protein per kg coffee beans, preferably 0.05-10 mg enzyme protein per kg coffee beans, more preferably 0.1-5 mg enzyme protein per kg coffee beans. After the soaking in the asparaginase solution, the coffee beans may be dried. The heat-treatment of the asparaginase treated food material may be performed by roasting or toasting to obtain the roasted coffee beans.

In another embodiment of the invention, the heat-treated food product is a coffee-based food product, e.g., roasted coffee beans or coffee obtained by extraction of the roasted coffee beans, and the food material which is to be contacted with asparaginase is a water extract of unroasted coffee beans. Prior to step (a), the unroasted coffee beans are subjected to an extraction in water, e.g., at a temperature of between 50 and 90° C. for a time of 3-12 hours, so as to obtain a water extract and extracted unroasted coffee beans. The water extract is separated from the extracted unroasted coffee beans. The extracted unroasted coffee beans may be dried, e.g., to a humidity of 10-30 wt. %. The contacting with asparaginase is performed by adding the asparaginase to the water extract at a temperature of 60-110° C. and allowing the asparaginase to react, e.g., for between 10 minutes and 2 hours. Optionally, the water extract may be decaffeinated prior to, at the same time or after the asparaginase treatment. The asparaginase treated was ter extract may be concentrated. After step (a) but before step (b), the optionally concentrated asparaginase treated water extract is reincorporated in the extracted unroasted coffee beans, which have optionally been dried, to obtain wet reincorporated unroasted coffee beans. The wet reincorporated unroasted coffee beans may be dried to obtain dry reincorporated unroasted coffee beans having a humidity of, e.g., 8-12.5 wt. %. The heat-treatment of the asparaginase treated food material may be performed by roasting or toasting the reincorporated unroasted coffee beans to obtain the roasted coffee beans.

Food products obtained by a method of the invention are characterized by significantly reduced acrylamide levels in comparison with equivalent food products obtainable by a production method that does not comprise adding an asparaginase in an amount that is effective in reducing the level of asparagine involved in the formation of acrylamide during a heating step.

In another aspect, the invention provides food products obtainable by a method of the invention as described above.

### Asparaginase

An asparaginase in the context of the present invention means an enzyme having asparaginase activity, i.e., an enzyme that catalyzes the hydrolysis of asparagine to aspartic acid (EC 3.5.1.1).

Asparaginase activity may be determined according to one of the asparaginase activity assays described under Materials and Methods in the Examples, e.g., by the ASNU assay. In one embodiment, an asparaginase to be used in the method of the present invention has at least 20%, e.g., at least 40%, at least 50%, at least 60%, at least 70%, at least 80%, at least 90%, at least 95%, or at least 100% of the asparaginase activity of the mature polypeptide of SEQ ID NO: 10 when measured at pH 7 and at 37° C.

Asparaginase activity may also be determined, e.g., according to the phenol activity assay. This assay may be better for determining the asparaginase activity of a thermostable asparaginase. In one embodiment, an asparaginase to be used in the method of the present invention is a thermostable asparaginase having at least 20%, e.g., at least 40%, at least 50%, at least 60%, at least 70%, at least 80%, at least 90%, at least 95%, or at least 100% of the asparaginase activity of the mature polypeptide of SEQ ID NO: 10 when measured at 70° C. and pH 7 according to the phenol activity assay described under Materials and Methods in the Examples.

The asparaginase activity may be determined per microgram asparaginase enzyme.

In a preferred embodiment, the asparaginase is a thermostable asparaginase.

A thermostable enzyme in the context of the present invention may be defined as an asparaginase, which after incubation at 70° C. for 60 minutes has a residual activity of at least 75%. The residual activity may be measured according to the phenol activity assay described under Materials and Methods in the Examples.

In a more preferred embodiment, the asparaginase is a hyperthermostable asparaginase.

A hyperthermostable asparaginase in the context of the present invention may be defined as an asparaginase, which after incubation at 80° C. for 60 minutes has a residual activity of at least 75%. The residual activity may be measured according to the phenol activity assay described under Materials and Methods in the Examples. A hyperthermostable asparaginase may have a denaturation temperature determined by Differential Scanning calorimetry (DSC) at pH 7 of at least 90° C. In one embodiment, a hyperthermostable asparaginase in the context of the present invention is an asparaginase which originates from an organism belonging to the domain Archaea.

The asparaginase may be obtained from a microorganism, preferably from an archaeon or from a thermophilic bacterium. For purposes of the present invention, the term “obtained from” as used herein in connection with a given source shall mean that the asparaginase encoded by a polynucleotide is produced by the source or by a strain in which the polynucleotide from the source has been inserted.

It may be a wild type asparaginase, i.e., an asparaginase found in nature, or it may be a variant asparaginase, i.e., an asparaginase comprising an alteration, i.e., a substitution, insertion, and/or deletion, at one or more (e.g., several) positions compared to a parent asparaginase from which it may have been derived. A substitution means replacement of the amino acid occupying a position with a different amino acid; a deletion means removal of the amino acid occupying a position; and an insertion means adding an amino acid adjacent to and immediately following the amino acid occupying a position.

In a preferred embodiment, the asparaginase or its parent, preferably the asparaginase, is obtained from an archaeon in the family Thermococcaceae, preferably of the genus Thermococcus, more preferably of the species Thermococcus kodakaraensis.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of thermophilic bacteria or archaea are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The asparaginase may be identified and obtained from other sources including microorganisms isolated from nature (e.g., soil, composts, water, etc.) or DNA samples obtained directly from natural materials (e.g., soil, composts, water, etc.). Techniques for isolating microorganisms and DNA directly from natural habitats are well known in the art. A polynucleotide encoding the asparaginase may then be obtained by similarly screening a genomic DNA or cDNA library of another microorganism or mixed DNA sample. Once a polynucleotide encoding an asparaginase has been detected, the polynucleotide can be isolated or cloned by utilizing techniques that are known to those of ordinary skill in the art (see, e.g., Sambrook et al., 1989, Molecular Cloning, A Laboratory Manual, 2d edition, Cold Spring Harbor, N.Y.).

In a preferred embodiment, the asparaginase has at least 60% sequence identity to the mature polypeptide of SEQ ID NO: 10, such as at least 70%, at least 80%, at least 90%, at least 95% or at least 98% sequence identity to the mature polypeptide of SEQ ID NO: 10.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

In another preferred embodiment, the asparaginase comprises at most 10, e.g., at most 5, at most 4, at most 3, at most 2 or at most 1 amino acid differences compared to the mature polypeptide of SEQ ID NO: 10.

In another preferred embodiment, the asparaginase has an amino acid sequence which comprises the sequence of the mature polypeptide of SEQ ID NO: 10. In a more preferred embodiment, the asparaginase has an amino acid sequence which consists of the sequence of the mature polypeptide of SEQ ID NO: 10.

Polynucleotides and Expression from these

In another aspect, the invention relates to an isolated polynucleotide having at least 60% sequence identity to SEQ ID NO: 9, such as at least 70%, at least 80%, at least 90%, at least 95%, at least 97%, at least 98% or at least 99% sequence identity to SEQ ID NO: 9, which encodes an asparaginase.

The invention also relates to a nucleic acid construct comprising such polynucleotide operably linked to one or more control sequences that direct the production of the asparaginase in an expression host. In a preferred embodiment, the expression host is a bacterial host cell. In a more preferred embodiment, the expression host is a Bacillus strain.

A polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, Molecular Cloning, A Laboratory Manual, 2d edition, Cold Spring Harbor, N.Y. Examples of tandem promoters are disclosed in WO 99/43835.

The control sequence may also be a transcription terminator, which is recognized by a host cell to terminate transcription. The terminator is operably linked to the 3′ terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′ terminus of the polynucleotide encoding the polypeptide. Any leader that is functional in the host cell may be used.

The control sequence may also be a polyadenylation sequence, a sequence operably linked to the 3′ terminus of the polynucleotide and, when transcribed, is recognized by the host cell as a signal to add polyadenosine residues to transcribed mRNA. Any polyadenylation sequence that is functional in the host cell may be used.

The control sequence may also be a signal peptide coding region that encodes a signal peptide linked to the N terminus of a polypeptide and directs the polypeptide into the cell's secretory pathway. The 5′ end of the coding sequence of the polynucleotide may inherently contain a signal peptide coding sequence naturally linked in translation reading frame with the segment of the coding sequence that encodes the polypeptide. Alternatively, the 5′ end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N terminus of a polypeptide and the signal peptide sequence is positioned next to the N terminus of the propeptide sequence.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. Other examples of regulatory sequences are those that allow for gene amplification.

The invention also relates to an expression vector comprising the polynucleotide of the invention operably linked to one or more control sequences that direct the production of the asparaginase in an expression host. In a preferred embodiment, the expression host is a bacterial host cell. In a more preferred embodiment, the expression host is a Bacillus strain.

The control sequences may include a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

The invention also relates to a recombinant host cell comprising such polynucleotide operably linked to one or more control sequences that direct the production of the asparaginase.

A construct or vector comprising a polynucleotide is introduced into a host cell so that the construct or vector is maintained as a chromosomal integrant or as a self-replicating extrachromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryotic host cell, which may be a Gram-positive or a Gram-negative bacterium. Preferably, the host cell is a Gram-positive bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces.

In a preferred embodiment, the recombinant host cell is a recombinant Bacillus host cell.

The Bacillus host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). However, any method known in the art for introducing DNA into a Bacillus host cell can be used.

The invention also relates to a method of producing an asparaginase, comprising:

(a) cultivating such host cell under conditions conducive for production of the asparaginase; and

(b) recovering the asparaginase.

The host cells are cultivated in a nutrient medium suitable for production of the asparaginase using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the asparaginase to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the asparaginase is secreted into the nutrient medium, the asparaginase can be recovered directly from the medium. If the asparaginase is not secreted, it can be recovered from cell lysates.

### Preferred Embodiments

1. A method for producing a heat-treated food product comprising:
   (a) contacting of a food material with an asparaginase which (i) has at least 60% sequence identity to the mature polypeptide of SEQ ID NO: 10, (ii) is encoded by a polynucleotide having at least 60% sequence identity to SEQ ID NO: 9, or (iii) is a variant of the mature polypeptide of SEQ ID NO: 10 comprising a substitution, deletion, and/or insertion at one or more positions; and
   (b) heat-treating the asparaginase treated food material to obtain the heat-treated food product.
2. The method of embodiment 1, wherein the asparaginase has at least 60% sequence identity to the mature polypeptide of SEQ ID NO: 10, such as at least 70%, at least 80%, at least 90%, at least 95%, at least 97%, at least 98% or at least 99% sequence identity to the mature polypeptide of SEQ ID NO: 10.
3. The method of embodiment 1, wherein the asparaginase is encoded by a polynucleotide having at least 60% sequence identity to SEQ ID NO: 9, such as at least 70%, at least 80%, at least 90%, at least 95%, at least 97%, at least 98% or at least 99% sequence identity to SEQ ID NO: 9.
4. The method of embodiment 1, wherein the asparaginase is a variant of the mature polypeptide of SEQ ID NO: 10 comprising a substitution, deletion, and/or insertion at one or more positions.
5. The method of any of the preceding embodiments, wherein the asparaginase is a thermostable asparaginase, preferably a hyperthermostable asparaginase].
6. The method of any of the preceding embodiments, wherein in step (a), the asparaginase is added to the food material at a temperature of at least 60° C., such as at least 80° C.
7. The method of any of the preceding embodiments, wherein in step (a), the asparaginase is added to the food material at a temperature of 60-110° C. such as at a temperature of 80-105° C.
8. The method of any of the preceding embodiments wherein the contacting in step (a) is for at least 2 minutes.
9. The method of any of the preceding embodiments, wherein the contacting in step (a) is for between 1 minute and 3 hours, preferably for between 2 minutes and 2 hours.
10. The method of any of the preceding embodiments, wherein the asparaginase is obtained from Thermococcus, preferably from Thermococcus kodakaraensis.
11. The method of any of the preceding embodiments, wherein the heat-treated food product is French fries, and wherein the food material is blanched potato strips.
12. The method of embodiment 11, wherein step (a) is performed by blanched potato strips being dipped in, incubated in or sprayed with a solution of the asparaginase at a temperature of 60-95° C., such as at a temperature of 65-75° C., and wherein the potato strips are dried after step (a) and before step (b).
13. The method of any of embodiments 1-10, wherein the heat-treated food product is sliced potato chips, and wherein the food material is potato slices.
14. The method of embodiment 13, wherein step (a) is performed by potato slices being blanched for 1-10 minutes at a temperature of 60-100° C., such as a temperature of 65-85° C., in an aqueous solution comprising the asparaginase.
15. The method of any of embodiments 1-10, wherein the heat-treated food product is a potato-based food product and wherein the food material to be treated with asparaginase is mashed potato, a potato-based dough or a suspension of a dehydrated potato product, such as potato flakes or granules.
16. The method of embodiment 15, wherein step (a) is performed by blending the asparaginase into a potato material selected among mashed potato, a potato-based dough or a suspension of a dehydrated potato product, such as potato flakes or granules, at a temperature of 60-100° C., preferably 80-100° C., more preferably 90-95° C.
17. The method of any of embodiments 1-10, wherein the heat-treated food product is a breakfast cereal and wherein the food material comprises whole or processed cereal kernels or grains.
18. The method of embodiment 17, wherein step (a) is performed by blending the asparaginase into a food material comprising whole wheat flour, wheat flour, oat flour, corn flour, rice flour, rye flour, wheat kernels, oat kernels or oat flakes at a temperature of 60-110° C., preferably 80-105° C.
19. The method of any of embodiments 1-10, wherein the heat-treated food product is roasted coffee beans and wherein the food material is unroasted coffee beans or a water extract of unroasted coffee beans.
20. The method of embodiment 19, wherein step (a) is performed by unroasted coffee beans, which have optionally been steamed, being soaked in a solution comprising asparaginase at a temperature of 60-110° C.
21. The method of embodiment 19, wherein prior to step (a), unroasted coffee beans are subjected to an extraction in water so as to obtain a water extract and extracted unroasted coffee beans, and the water extract is separated from the extracted unroasted coffee beans; wherein the extracted unroasted coffee beans are dried; wherein step (a) is performed by adding the asparaginase to the water extract at a temperature of 60-110° C. and allowing the asparaginase to react for between 10 minutes and 2 hours; wherein after step (a) but before step (b), the asparaginase treated water extract is concentrated and reincorporated in the dried extracted unroasted coffee beans to obtain wet reincorporated unroasted coffee beans; wherein the wet reincorporated unroasted coffee beans are dried to obtain dry reincorporated unroasted coffee beans; and wherein step (b) comprises toasting the dry reincorporated unroasted coffee beans to obtain roasted coffee beans.
22. Use of an asparaginase in the production of a heat-treated food product, where the asparaginase (i) has at least 60% sequence identity to the mature polypeptide of SEQ ID NO: 10, (ii) is encoded by a polynucleotide having at least 60% sequence identity to SEQ ID NO: 9, or (iii) is a variant of the mature polypeptide of SEQ ID NO: 10 comprising a substitution, deletion, and/or insertion at one or more positions.
23. Use of an asparaginase according to embodiment 22 in the production of a potato-based food product.
24. Use of an asparaginase according to embodiment 22 in the production of French fries.
25. Use of an asparaginase according to embodiment 22 in the production of sliced potato chips.
26. Use of an asparaginase according to embodiment 22 in the production of a breakfast cereal.
27. Use of an asparaginase according to embodiment 22 in the production of roasted coffee beans.
28. Use of an asparaginase for treatment of a food material, where the asparaginase (i) has at least 60% sequence identity to the mature polypeptide of SEQ ID NO: 10, (ii) is encoded by a polynucleotide having at least 60% sequence identity to SEQ ID NO: 9, or (iii) is a variant of the mature polypeptide of SEQ ID NO: 10 comprising a substitution, deletion, and/or insertion at one or more positions.
29. Use of an asparaginase according to embodiment 28 for treatment of a potato-based food material.
30. Use of an asparaginase according to embodiment 28 for treatment of mashed potato, a potato-based dough or a suspension of a dehydrated potato product, such as potato flakes or granules.
31. Use of an asparaginase according to embodiment 28 for treatment of cuts of potatoes.
32. Use of an asparaginase according to embodiment 28 for treatment of blanched potato strips.
33. Use of an asparaginase according to embodiment 28 for treatment of sliced potatoes.
34. Use of an asparaginase according to embodiment 28 for treatment of a food material which comprises whole or processed cereal kernels or grains.
35. Use of an asparaginase according to embodiment 28 for treatment of a food material which comprises whole wheat flour, wheat flour, oat flour, corn flour, rice flour, rye flour, wheat kernels, oat kernels or oat flakes.
36. Use of an asparaginase according to embodiment 28 for treatment of green coffee beans.
37. Use of an asparaginase according to any of embodiments 22-36, wherein the asparaginase has at least 60% sequence identity to the mature polypeptide of SEQ ID NO: 10, such as at least 70%, at least 80%, at least 90%, at least 95%, at least 97%, at least 98% or at least 99% sequence identity to the mature polypeptide of SEQ ID NO: 10.
38. Use of an asparaginase according to any of embodiments 22-36, wherein the asparaginase is encoded by a polynucleotide having at least 60% sequence identity to SEQ ID NO: 9, such as at least 70%, at least 80%, at least 90%, at least 95%, at least 97%, at least 98% or at least 99% sequence identity to SEQ ID NO: 9.
39. Use of an asparaginase according to any of embodiments 22-36, wherein the asparaginase is a variant of the mature polypeptide of SEQ ID NO: 10 comprising a substitution, deletion, and/or insertion at one or more positions.
40. Use of an asparaginase according to any of embodiments 22-36, wherein the asparaginase is a thermostable asparaginase, preferably a hyperthermostable asparaginase.
41. An isolated polynucleotide having at least 60% sequence identity to SEQ ID NO: 9, such as at least 70%, at least 80%, at least 90%, at least 95%, at least 97%, at least 98% or at least 99% sequence identity to SEQ ID NO: 9, which encodes an asparaginase.
42. A nucleic acid construct or expression vector comprising the polynucleotide of embodiment 41 operably linked to one or more control sequences that direct the production of the asparaginase in an expression host.
43. The nucleic acid construct or the expression vector of embodiment 42, wherein the expression host is a Bacillus strain.
44. A recombinant host cell comprising the polynucleotide of embodiment 41 operably linked to one or more control sequences that direct the production of the asparaginase.
45. The recombinant host cell of embodiment 44 which is a recombinant Bacillus host cell.
46. A method of producing an asparaginase, comprising:
   (a) cultivating the host cell of embodiment 44 under conditions conducive for production of the asparaginase; and
   (b) recovering the asparaginase.
47. The method of embodiment 46 which comprises cultivating the recombinant Bacillus host cell of embodiment 45.

### EXAMPLES

### Materials and Methods 1

Water is Milli-Q® water where nothing else is specified.

### Asparaginase Activity (ASNU) Assay

The activity of asparaginase may be measured in ASNU. An asparaginase unit (ASNU) is defined as the amount of enzyme needed to generate 1.0 micromole of ammonia in 1 minute at 37° C. and pH 7.0, in 0.1 M MOPS buffer with 9.2 mg/ml L-asparagine.

Asparaginase hydrolyzes L-asparagine to aspartic acid and ammonium. The produced ammonium is combined with α-ketoglutarate to form glutamic acid whereby NADH is oxidized to NAD+. The reaction is catalysed by a surplus of glutamate dehydrogenase. The consumption of NADH is measured by photometry at 340 nm. NADH has an absorbance at 340 nm, while NAD+ has no absorbance. A decrease in color is thus measured, and can be correlated to asparaginase activity.

Activity is determined relative to an asparaginase standard of known activity. A commercial product having a declared activity like Acrylaway® may be used as standard.

### Phenol Activity Assay for Quantification of Thermostable Asparaginase

### Principle

Asparaginase activity was determined in two steps. The first step is an enzymatic step where ammonia is formed by the catalytic action of the asparaginase from L-asparagine. The second step is a non-enzymatic detection step wherein the formed ammonia is derivatized to a blue indophenol compound.

### Enzyme and Standard Incubation

Ammonium chloride was used as standard in the range of 0 mM to 10 mM. 20 μL ammonium standard or diluted asparaginase was incubated with 100 μL asparagine solution in a PCR machine at appropriate temperature, e.g., 70° C. for 10 min. The reaction was stopped by transferring the samples to an ice bath.

L-Asparagine solution: L-Asparagine (10 g/L) was dissolved in 100 mM assay buffer (100 mM sodium acetate, 100 mM phosphate, 100 mM borate and 0.01% Triton X-100 at pH 7 (pH was adjusted with HCl or NaOH)).

### Quantification

Three different color reagents are needed:

A—4% (w/v) Phenol, 0.015% (w/v) sodium pentacyanonitrosylferrate (III) dihydrate (Na₂[Fe(CN)₅NO].2H₂O),

B—5% (w/v) Potassium hydroxide, and

C—28% (w/v) Potassium carbonate, 6% (v/v) sodium hypochlorite.

The PCR plate from the ammonia formation step was spun for 5 minutes at 3000 rpm, 5° C. to remove condensation from the sealing tape. 10 μL was transferred to a MTP and diluted with 240 μL MQ water (shake for 1 minute at 750 rpm to mix).

60 μL of the samples was transferred to a new MTP. To each well, 60 μL of color reagent A was added (shake gently to mix). To each well, 30 μL of color reagent B was added (shake gently to mix). To each well, 60 μL of color reagent C was added (shake gently to mix). Carefully seal the plate and incubate for 20 minutes at 37° C., 750 rpm on an Eppendorf thermomixer equipped with an MTP adapter. The absorbance was measured at 630 nm. Absorbance of the standards were plotted as a function of NH₄⁺ concentration in the standards, and ammonia produced in the enzyme samples calculated by comparison to this curve. Activity is given as released NH₄⁺ per minute per ml sample.

### Example 1

### Cloning of Asparaginase from Thermococcus kodakaraensis Strain JCM 12380 in Bacillus Subtilis

The asparaginase gene sequence (SEQ ID NO: 1) originates from the strain Thermococcus kodakaraensis strain JCM 12380 which is commercially available from the Japan Collection of Microorganisms. The type strain was described by Atomi et al, (“Description of Thermococcus kodakaraensis sp. nov., a well studied hyperthermophilic archaeon previously reported as Pyrococcus sp. KOD1”, Archaea 1(4), 263-267, 2004). The deduced protein sequence (SEQ ID NO: 2; accession number SWISSPROT:Q5JIW4) shares 83% sequence identity by pairwise alignment to the asparaginase from Thermococcus sp. AM4 (SEQ ID NO: 3; accession number SWISSPROT:B7R1B5), 79% sequence identity to the asparaginase from Thermococcus gammatolerans(SEQ ID NO: 4; accession number SWISSPROT:C5A6T2), 65% sequence identity to the asparaginase from Thermococcus sibiricus (SEQ ID NO: 5; accession number SWISSPROT:C6A532), and 58% sequence identity to the asparaginase from Pyrococcus furiosus (SEQ ID NO: 6; accession number GENESEQP:AWF59717).

A synthetic gene based on the protein sequence of asparaginase from T. kodakaraensis was designed by optimizing the gene codon usage for B. subtilis as described in WO 2012/025577. For subcloning into the B. subtilis expression cassette, the gene sequence was amplified by PCR from the commercially purchased synthetized gene using the oligomers:

Fwd oligomer:
(SEQ ID NO: 7)
AAAGGAGAGGATAAAGAATGAAACTTCTTGTTCTTGGT
Reverse oligomer:
(SEQ ID NO: 8)
GCGTTTTTTTATTGATTAACGCGTAGAACCTGTGATTTCACCA

The optimized gene was fused to genetic expression elements as described in WO 99/43835 (hereby incorporated by reference). The (sub-)cloning principle by PCR is known to the person skilled in the art. The reverse oligomer (SEQ ID NO: 8) completed the CDS of the optimized gene by a stop codon, resulting in gene sequence SEQ ID NO: 9 which encodes amino acid sequence SEQ ID NO: 10 (identical to SEQ ID NO: 2). The gene construct was integrated by homologous recombination into the Bacillus subtilis host cell genome upon transformation. The B. subtilis expression host was deficient of the following gene products by gene insertion or gene deletion on its chromosome: SpollAC−, Biol−, NprE−, AprE−, AmyE−, SrfAC−, Bpr−, Vpr−, Epr−, IspA−. The gene construct was expressed under the control of a triple promoter system (as described in WO 99/43835). The gene coding for chloramphenicol acetyltransferase was used as maker (as described in Diderichsen et al., 1993, Plasmid 30: 312-315).

One expression clone was selected and was cultivated on a rotary shaking table in 500 mL baffled Erlenmeyer flasks each containing 100 mL casein based media supplemented with 34 mg/L chloramphenicol. The clone was cultivated for 6 days at 26° C. and successful expression was determined by SDS-PAGE analysis using cell free supernatant of the cultivated expression clone. For preparation of the sample for SDS-PAGE analysis the cell culture was centrifuged and the supernatant filtered through a 0.45 μm filter, followed by incubation at 80° C. for 20 min. in order to inactivate host cell proteases. Centrifugation was repeated to remove any precipitate formed during the heat treatment. Recombinant expression of the protein was detected as distinct protein band at approx. 36 kDa.

### Example 2

### Purification of Asparaginase from Thermococcus kodakaraensis

The harvested cell culture of Example 1 was centrifuged, followed by incubation of the supernatant at 70° C. for 20 min. in order to inactivate host cell proteases. Centrifugation was repeated to remove any precipitate formed during the heat treatment, and the supernatant filtered through a 0.45 μm filter. This was followed by a 0.22 μm filtration. The pH of the filtrate was adjusted to 5.75 and loaded onto a mixed-mode chromatography column (XpressLine Pro A from Upfront Chromatography NS) equilibrated in 100 mM MES pH 5.75, and eluted by a step gradient using a 100 mM Tris pH 9.0 buffer. Based on SDS-PAGE (reducing conditions), fractions containing protein of the expected Mw were pooled and buffer-exchanged into 25 mM HEPES pH 7.0 by dialysis using a 12-14 kDa MWCO membrane. This was followed by anion-exchange chromatography (packed bed of SOURCE™ 15Q; gradient: 0-100% B in 10 CV's; buffer A: 25 mM HEPES pH 7.0; buffer B: buffer A+1 M NaCl). Based on SDS-PAGE (reducing conditions), fractions containing protein of the expected Mw were pooled into two banches, differing in elution conditions (“low salt” vs. “high salt”) and these constituted the final products. Data reported here, refers to the batch eluting at low salt conditions in the final purification step.

In the capture step, a packed bed of Capto MMC from GE Healthcare Life Sciences could be used instead of the XpressLine Pro A from Upfront Chromatography NS.

### Materials and Methods 2

### Enzymes Used in the Following Examples:

Thermococcus kodakaraensis asparaginase encoded by a polynucleotide having the coding sequence shown as SEQ ID NO: 9 and purified according to Example 2.

Pyrococcus furiosus asparaginase disclosed in WO2008/151807.

### Example 3

### MS Intact and N-Terminal Sequencing

Intact molecular weight and N-terminal sequence of the T. kodakaraensis asparaginase was determined according to the following procedures:

### MS Intact

The intact molecular weight analyses were performed using a Bruker microTOF focus electrospray mass spectrometer (Bruker Daltonik GmbH, Bremen, DE). The samples were diluted to about 1 mg/ml in MQ water. The diluted samples were online washed on a MassPREP On-Line Desalting column (2.1×10 mm Part no. 186002785 Waters) and introduced to the electrospray source with a flow of 200 μl/h by an Agilent LC system. Data analysis is performed with DataAnalysis version 3.3 (Bruker Daltonik GmbH, Bremen, DE). The molecular weight of the samples was calculated by deconvolution of the raw data.

### N-Terminal Sequencing

The samples were prepared for SDS-PAGE and the resulting gels blotted on ProBlott PVDF membranes. Selected protein bands were cut out and placed in the blotting cartridge of an Applied Biosystems Procise protein sequencer. The N-terminal sequencing was carried out using the method run file for PVDF membrane samples (Pulsed liquid PVDF) according to the manufacturer's instructions. The N-terminal amino acid sequence can be deduced from the resulting chromatograms by comparing the retention time of the peaks in the chromatograms to the retention times of the PTH-amino-acids in the standard chromatogram.

### Results

Intact molecular weight: Two peaks were observed in the mass spectrum at 35675.7 Da; and 35690.8 Da. The latter peak may indicate oxidation. The calculated molecular weight for amino acids 1 to 328 of SEQ ID NO: 10 is 35674.7 Da.

The N-terminal sequence was determined as MKLLVLG.

### Example 4

### pH Activity of Thermococcus kodakaraensis Asparaginase

pH activity of the Thermococcus kodakaraensis asparaginase was evaluated by determining catalytic activity during incubation at selected pH's for 10 min. at 70° C. Initially, the samples were all diluted in 0.01% (w/v) of Triton™ X-100. Just before incubation, the diluted samples were mixed 1:1 with incubation buffer (200 mM acetate, 200 mM phosphate, 200 mM borate and 0.02% (w/v) Triton™ X-100 pH adjusted to 4.0, 5.0, 6.0, 7.0, 8.0, or 9.0) and treated as follows:

Incubation:
20 μL sample diluted in 0.01% (w/v) Triton™ X-100 (blank samples consisted of 20 μL 0.01% (w/v) Triton™ X-100) was transferred to a well in a PCR cycler followed by 50 μL sample buffer (200 mM acetate, 200 mM phosphate, 200 mM borate and 0.02% (w/v) Triton™ X-100 pH adjusted to 4.0, 5.0, 6.0, 7.0, 8.0, or 9.0)
Finally, 50 μL substrate solution (20 mg/ml L-asparagine dissolved in 0.01% (w/v) Triton™ X-100) was added, the plate sealed, and incubated for 10 min. at 70° C.
Following incubation, all samples were rapidly cooled to 5° C., using the cooling option in the PCR cycler, in order to stop the reaction. After cooling down, the samples were centrifuged in order to precipitate any condensed liquid on the sealing tape

The activity assay consisted of two separate (de-coupled) parts:
1) An enzymatic step where ammonia was formed by the catalytic action of the asparaginase (corresponding to the incubation step described above)
2) A non-enzymatic detection step wherein the formed ammonia was derivatized to a blue indophenol compound with an absorption maximum at 630 nm

Activity assay—detection of ammonia (color reaction):
10 μL sample was diluted by 240 μL ultrapure water in a microtiter plate
60 μL of the above solution was transferred to a new MTP plate and mixed with 60 μL color reagent A (4% (w/v) Phenol, 0.015% (w/v) sodium pentacyanonitrosylferrate (III) dihydrate (Na₂[Fe(CN)₅NO].2H₂O)).
30 μL color reagent B (5% (w/v) KOH) was added
Finally, 60 μL color reagent C (28% (w/v) potassium carbonate, 6% (v/v) sodium hypochlorite (Sigma-Aldrich 239305-25 ml, <5% available Cl₂) was added, the plate sealed, and incubated at 37° C. for 20 minutes with mixing
Endpoint was measured at 630 nm

TABLE 1
Relative activity (%)

| pH | P. furiosus | T. kodakaraensis |
|---|---|---|
| 4 | 21 | 2 |
| 5 | 47 | 34 |
| 6 | 62 | 50 |
| 7 | 74 | 67 |
| 8 | 85 | 84 |
| 9 | 100 | 100 |

The catalytic activity of the Thermococcus kodakaraensis asparaginase increased as a function of pH in the investigated interval, starting from almost no activity at pH 4. In contrast, the Pyrococcus furiosus asparaginase displayed significant relative activity at pH 4 which also increased as a function of pH.

### Example 5

### pH Stability of Thermococcus kodakaraensis Asparaginase

pH stability of the Thermococcus kodakaraensis asparaginase was evaluated by determining residual activity after incubation for 60 min. at pH 7 and selected pH's in the interval 4-9. Initially, the samples were all diluted in 0.01% (w/v) of Triton™ X-100. Just before incubation, the diluted samples were mixed 1:1 with incubation buffer (200 mM acetate, 200 mM phosphate, 200 mM borate and 0.02% (w/v) Triton™ X-100 pH adjusted to 4.0, 5.0, 6.0, 7.0, 8.0, or 9.0) and treated as follows:

Incubation:
100 μL sample mix was transferred to a PCR cycler, sealed, and incubated for 60 min. at 70° C. Reference sample was prepared in incubation buffer pH 7.0 and stored at 4° C.
Following incubation, all samples were diluted 10-fold in assay buffer (100 mM acetate, 100 mM phosphate, 100 mM borate and 0.01% (w/v) Triton™ X-100 pH 7.0) prior to the activity assay

The activity assay consisted of two separate (de-coupled) parts:
3) An enzymatic step where ammonia was formed by the catalytic action of the asparaginase
4) A non-enzymatic detection step wherein the formed ammonia was derivatized to a blue indophenol compound with an absorption maximum at 630 nm

Activity assay—formation of ammonia:
20 μL sample was added to each well in a PCR plate
100 μL substrate solution (10 mg/ml L-asparagine in assay buffer) was added
The plate was sealed and incubated for 10 min. at 70° C. on a PCR cycler
The reaction was stopped by transfer to an ice bath. After cooling down, the samples were centrifuged in order to precipitate any condensed liquid on the sealing tape

Activity assay—detection of ammonia (color reaction):
10 μL sample was diluted by 240 μL ultrapure water in a microtiter plate
60 μL of the above solution was transferred to a new MTP plate and mixed with 60 μL color reagent A (4% (w/v) Phenol, 0.015% (w/v) sodium pentacyanonitrosylferrate (III) dihydrate (Na₂[Fe(CN)₅NO].2H₂O)).
30 μL color reagent B (5% (w/v) KOH) was added
Finally, 60 μL color reagent C (28% (w/v) potassium carbonate, 6% (v/v) sodium hypochlorite (Sigma-Aldrich 239305-25 ml, <5% available Cl₂) was added, the plate sealed, and incubated at 37° C. for 20 minutes with mixing
Endpoint was measured at 630 nm

TABLE 2
Relative activity (%)

| pH | P. furiosus | T. kodakaraensis |
|---|---|---|
| 4 | 87 | 68 |
| 5 | 83 | 94 |
| 6 | 94 | 103 |
| 7 | 107 | 108 |
| 8 | 99 | 104 |
| 9 | 109 | 106 |
| Ref. | 100 | 100 |

The residual activity of the T. kodakaraensis asparaginase dropped to less than 80% after incubation at 70° C. for 60 min. at pH 4. In contrast, the P. furiosus asparaginase retained more than 80% residual activity under these conditions.

### Example 6

### Thermostability of Thermococcus kodakaraensis Asparaginase Evaluated by DSC

Thermostability of the Thermococcus kodakaraensis asparaginase was evaluated by Differential Scanning calorimetry (DSC) at pH 5 and 7. The temperature, corresponding to the apex of the peak in the thermogram, was noted as the denaturation temperature, T_{d} (° C.). The purified batch of asparaginase was buffer-exchanged to the appropriate buffer solution (pH 5: 50 mM acetate; pH 7: 50 mM HEPES) by gravity flow in a small desalting column (e.g. NAP™-5). Following buffer-exchange, the non-ionic surfactant Triton™ X-100 was added to a concentration of 100 ppm. Final asparaginase concentration was approx. 0.4-0.5 mg/ml, except for pH 5, where T. kodakaraensis asparaginase was at a concentration of approx. 0.8 mg/ml. The sample was analyzed by a MicroCal VP-Capillary DSC system at a scan rate of 200 K/h. Denaturation temperatures of 107.2° C. (pH 5) and 110.7° C. (pH 7) were observed. In comparison, at similar conditions, the observed T_{d}'s of the P. furiosus asparaginase were 107.3° C. (pH 5) and 111.1° C. (pH 7).

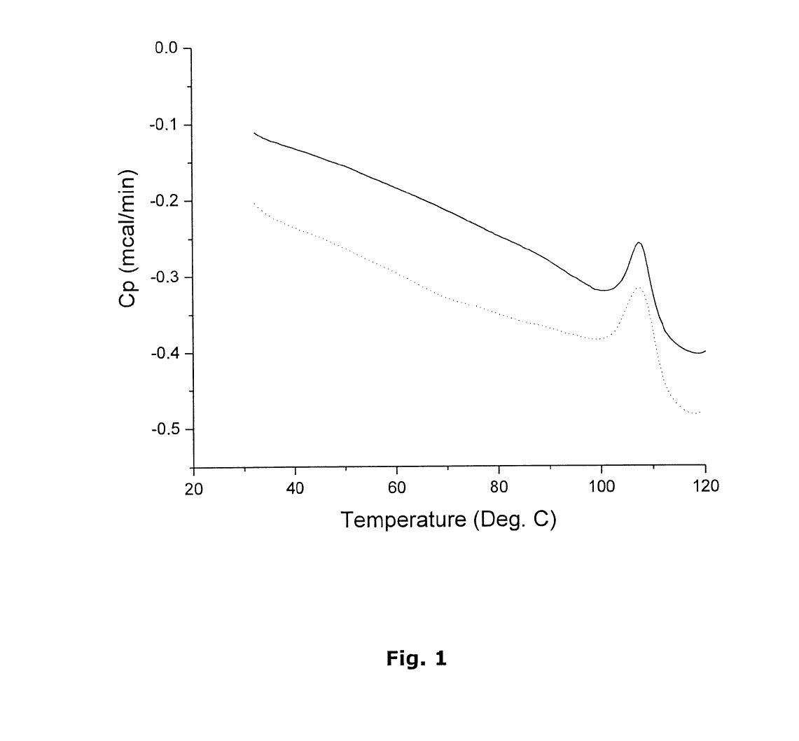

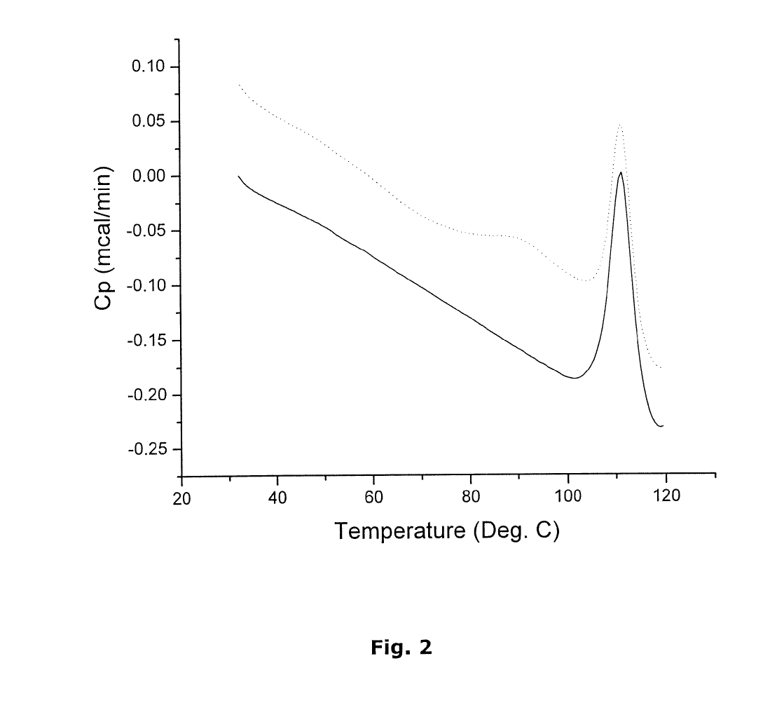

The DSC thermograms are shown in FIG. 1 (pH 5) and FIG. 2 (pH 7). Dotted curve: Thermococcus kodakaraensis asparaginase; solid curve: Pyrococcus furiosus asparaginase.

### Example 7

### Thermostability of Thermococcus kodakaraensis Asparaginase Evaluated by Residual Activity

Thermostability of the Thermococcus kodakaraensis asparaginase was evaluated by determining residual activity after incubation for 60 min. at pH 7 and selected temperatures in the interval 40-90° C. The samples were all diluted in assay buffer (100 mM acetate, 100 mM phosphate, 100 mM borate and 0.01% (w/v) Triton™ X-100 pH 7.0) and treated as follows:

Incubation:
50 μL sample was transferred to a PCR cycler, sealed, and incubated for 60 min. at 40, 50, 60, 70, 80, or 90° C. Reference sample was stored at 5° C.
Following incubation, all samples were diluted 10-fold in assay buffer prior to the activity assay

The activity assay consisted of two separate (de-coupled) parts:
5) An enzymatic step where ammonia was formed by the catalytic action of the asparaginase
6) A non-enzymatic detection step wherein the formed ammonia was derivatized to a blue indophenol compound with an absorption maximum at 630 nm

Activity assay—formation of ammonia:
20 μL sample was added to each well in a PCR plate
100 μL substrate solution (10 mg/ml L-asparagine in assay buffer) was added
The plate was sealed and incubated for 10 min. at 70° C. on a PCR cycler
The reaction was stopped by transfer to an ice bath. After cooling down, the samples were centrifuged in order to precipitate any condensed liquid on the sealing tape

Activity assay—detection of ammonia (color reaction):
10 μL sample was diluted by 240 μL ultrapure water in a microtiter plate
60 μL of the above solution was transferred to a new MTP plate and mixed with 60 μL color reagent A (4% (w/v) Phenol, 0.015% (w/v) sodium pentacyanonitrosylferrate (III) dihydrate (Na₂[Fe(CN)₅NO].2H₂O)).
30 μL color reagent B (5% (w/v) KOH) was added
Finally, 60 μL color reagent C (28% (w/v) Potassium carbonate, 6% (v/v) sodium hypochlorite (Sigma-Aldrich 239305-25 ml, <5% available Cl₂) was added, the plate sealed, and incubated at 37° C. for 20 minutes with mixing
Endpoint was measured at 630 nm

TABLE 3
Relative activity (%)

| Temperature (° C.) | P. furiosus | T. kodakaraensis |
|---|---|---|
| 40 | 113 | 99 |
| 50 | 111 | 98 |
| 60 | 107 | 104 |
| 70 | 105 | 110 |
| 80 | 106 | 99 |
| 90 | 102 | 106 |
| Ref. | 100 | 100 |

Both the P. furiosus and the T. kodakaraensis asparaginase retain their activity in the investigated temperature interval.

### Materials and Methods 3

### Methods Used in the Following Examples:

Quantification of Acrylamide in Samples Extracted from Food.

Acrylamide was routinely quantified by a combined method of high pressure ion exclusion chromatography and mass spectrometry.

Analysis was performed on a Thermo Fischer Ion chromatography system 5000, comprising an auto sampler with cooling option (AS-AP), a gradient high pressure pump (GP), a column compartment with temperature control (DC), a single wavelength UV detector (VWD) and a single quadrupole mass spectrometer (MSQ plus).

The ion exclusion column Dionex IonPac ICE-AS1 (4×50 mm) was equilibrated with 3 mM formic acid in a 60:40 mixture of milli-Q water and acetonitrile and the flow rate was set to 100 μL/minute. Samples were stored on 8° C. prior to analysis; an aliquot of 50 μL was injected by an automated sampler, the isocratic chromatographic separation was monitored for control by UV absorbance. Prior to infusion into the mass spectrometer the eluent was diluted with 150 μL/min of 1:1 mixture of acetonitrile and water delivered by an auxiliary pump, this ensured a stable flow for the electrospray ionization in positive ion mode in the mass spectrometer. The ion count in the mass range from 71.6 Dalton to 72.3 Dalton was collected as a chromatogram and the acrylamide was quantified by peak integration. Peak areas were compared to peak areas of acrylamide standards in the concentration range from 20 ppb to 500 ppb.

### Quantification of Asparagine and Aspartic Acid Using HPLC

Asparagine and aspartic acid content of samples were analyzed on a ThermoFisher WPS3000 high pressure liquid chromatography system comprising of a quaternary pump, an auto sampler with temperature control, a column oven and a tunable fluorescence detector.

Samples were analyzed after automated pre-column derivatisation. 30 μL milli-Q water, 10 μL of 0.4 M borate buffer pH 10.2, 2 μL sample, and 2 μL ortho-phthalaldehyde 10 g/L in 0.4 M borate buffer pH 10.2 were collected and mixed by pipetting up and down in a mixing vial; 100 μL milliQ water was added, and 2 μL was finally injected for chromatographic analysis on an Agilent zorbax eclipse AAA column (4.6 mm by 150 mm, 3.5 μm particle size) with the corresponding guard column.

The pump was set to a constant flow rate of 2 ml/minute, the column was initially equilibrated with 20 mM phosphate buffer pH 7.5 and asparagine was eluted with a linear gradient from 4 minutes to 12 minutes from 0% to 100% of a 45% methanol 45% acetonitrile 10% water mixture. Fluorescence of the asparagine derivative was excited with light at 340 nm and emission was quantified at 450 nm. Samples were analyzed by comparison to standard aspartic acid and asparagine in the concentration range from 0.05 mM to 0.75 mM.

### Example 8

### Enzyme Performance in Breakfast Cereal Model

Enzyme performance was tested in a breakfast cereal model lab set-up using 65.4 g whole wheat flour mixed with 1.3 g glucose syrup and 33.3 g water and asparaginase. The dough was mixed for 2 min using a handheld mixer, split in 3 equal bits and packed in a roasting bag (to avoid dry-out) and quickly heated to 95° C. for 52 sec using a microwave oven. The dough was kept in the roasting bag and incubated in a heating chamber at 95-100° C. for 25 min to mimic a batch steam-cooking process. After incubation each bit of dough was grinded in a coffee mill for 30 sec and mixed with a known amount (between 130-140 ml) of 0.05 N HCl to inactivate the enzyme. The samples were homogenised and centrifuged and asparagine and aspartic acid content in the supernatant analysed using HPLC as described above. Results are shown below.

TABLE 4
Asparagine and aspartic acid content as a function of enzyme
dosage in breakfast cereal dough after incubation at 95-100° C.
for 25 min. Enzyme used is the asparaginase from Pyrococcus furiosus

| Enzyme dosage | Asparagine (%) | Aspartic acid (%) |
|---|---|---|
| mg ep/kg flour | Asn/(asn + asp)*100 | Asp/(asn + asp)*100 |
| 0 | 80.1 | 19.9 |
| 0.2 | 51.4 | 48.6 |
| 0.4 | 15.9 | 84.1 |
| 0.8 | 3.8 | 96.2 |

TABLE 5
Asparagine and aspartic acid content as a function of enzyme
dosage in breakfast cereal dough after incubation at 95-100° C.
for 25 min. Enzyme used is the asparaginase from
Thermococcus kodakaraensis

| Enzyme dosage | Asparagine (%) | Aspartic acid (%) |
|---|---|---|
| mg ep/kg flour | Asn/(asn + asp)*100 | Asp/(asn + asp)*100 |
| 0 | 63.2 | 36.8 |
| 0.22 | 18.7 | 81.3 |
| 0.43 | 0.6 | 99.4 |
| 0.66 | 1.6 | 98.4 |

As seen from the results in Tables 4 and 5, the asparaginase from T. kodakaraensis is clearly more efficient than the enzyme from P. furiosus when comparing on an enzyme protein basis. 0.2 mg enzyme protein of the P. furiosus enzyme hydrolyse 29% asparagine, while a similar amount of the T. kodakaraensis enzyme hydrolyse 45% asparagine during 25 min incubation.

### Example 9

### Enzyme Performance in Potato Mash

Enzyme performance was tested in potato mash using a 10% dry matter slurry of rehydrated potato flakes in MQ water. The mash was pre-heated to 90° C. before enzyme addition. The mash was incubated at 90° C. for 30 min. Mixing was done manually every 5 min and samples taken every 5 to 10 min. 2 g samples are withdrawn and mixed with 8 ml 0.1 N HCL to inactivate the enzyme. The samples were mixed for 30 min and centrifuged, and asparagine and aspartic acid content analysed in the supernatant using HPLC as described above. Results are shown below.

TABLE 6
Asparagine and aspartic acid content in potato mash as a
function of time incubated at 90° C. Enzyme dosage was 3 mg
enzyme protein/kg DS. Enzyme used is the asparaginase from
Pyrococcus furiosus

| Time | Asparagine (%) | Aspartic acid (%) |
|---|---|---|
| min | Asn/(asn + asp)*100 | Asp/(asn + asp)*100 |
| 0 | 79 | 21 |
| 5 | 48 | 52 |
| 10 | 30 | 70 |
| 20 | 7 | 93 |
| 30 | 2 | 98 |

TABLE 7
Asparagine and aspartic acid content in potato mash
as a function of time incubated at 90° C. Enzyme dosage was
1.6 mg enzyme protein/kg DS. Enzyme used is the asparaginase
from Thermococcus kodakaraensis

| Time | Asparagine (%) | Aspartic acid (%) |
|---|---|---|
| min | Asn/(asn + asp)*100 | Asp/(asn + asp)*100 |
| 0 | 82 | 18 |
| 5 | 55 | 45 |
| 10 | 34 | 66 |
| 15 | 18 | 82 |
| 20 | 8 | 92 |
| 30 | 5 | 95 |

As seen from the results in Table 6 and 7, the asparaginase from T. kodakaraensis is clearly more efficient than the enzyme from P. furiosus, i.e. only 1.6 mg enzyme protein of the asparaginase from T. kodakaraensis is needed to match performance of 3 mg enzyme protein of the asparaginase from P. furiosus.

### Example 10

### Enzyme Performance in Sliced Potato Chips

### Sliced Potato Chip Procedure

Chipping potatoes (Lady Claire) were peeled with a potato peeler (OBH Nordica, Potato King, type 6770) and placed into a slicer (Robot Coupe® R301 Ultra, 2 mm slicer). The potato slices from the individual potatoes were mixed and held in de-ionized water until use (30 min). The potato blanching and the enzyme incubation were done in a 600 mL glass beaker which was placed in a temperature controlled water bath (IKA-Werke HBR 4 digital). The blanching/enzyme treatment was conducted at a temperature of 80° C. and at an incubation time of 3.5 min; the enzyme was applied 2 min prior to the addition of the potatoes. 40 g potato slices were weighed out and added to 400 mL heated deionized water. After the incubation, the blanched potato slices were collected using a sieve and added to a frying bath. The samples were fried for 260 s at 175° C. The enzyme dosage is calculated in mg EP/L blanching water and a dosage of ˜1 mg EP/L and ˜10 mg EP/L was tested. The enzyme trials were done in duplicates, while 6 controls (no enzyme) were included during the testing.

### Acrylamide Extraction and Analysis

The sliced potato chips were blended for 10 s at 10.000 rpm (Retsch GM200). 2 g of the blended crisps was mixed with 20 mL MQ water and the sample was homogenized using an Ultraturrax (IKA) for 1 min at 8000 rpm. Afterwards the sample was shaken for 60 min in a rotator followed by centrifugation at 3500 rpm for 15 min. Centrifugation divides the sample tube in 3 zones. 1.5 mL was taken from the middle zone and treated with amylase (BAN 480L) for starch removal (final amylase concentration 0.25 KNU/mL extract) and 15 μL Carrez solution I (potassium hexacyanoferrate(II) trihydrate (K₄[Fe(CN)₆]×3H₂O), 15 g/100 mL) per 1.5 mL extract and 15 μL Carrez solution II (zinc sulfate heptahydrate (ZnSO₄×7H₂O), 30 g/100 mL) per 1.5 mL extract for removal of co-extracted colloids. Following addition of amylase, Carrez I and Carrez II, the sample was left overnight in the fridge and the next day the sample was centrifuged at 20.000 g for 10 min and the supernatant was filtered using 0.22 μm prior to LC-MS analysis as described above.

The acrylamide results are presented below.

TABLE 8
Calculated reduction in acrylamide formation in final
sliced potato chips treated with asparaginase from Pyrococcus furiosus
and Thermococcus kodakaraensis at 2 different dosages
and incubated at 80° C. for 3.5 min. Reduction is calculated
by comparing to an average control sample without enzyme.

| | Acrylamide reduction |
|---|---|
| Sample, Dosage | vs Control sample, % |
| Control | 0 |
| Pyrococcus furiosus, 1 mgEP/L | 6 |
| Pyrococcus furiosus, 10 mgEP/L | 24 |
| Thermococcus kodakaraensis, 1 mgEP/L | 22 |
| Thermococcus kodakaraensis, 10 mgEP/L | 20 |

For Pyrococcus furiosus, the achieved acrylamide reduction was 6% reduction at 1 mgEP/L, 24% at 10mgEP/L.

Acrylamide levels in the final sliced potato chip treated with asparaginase from Thermococcus kodakaraensis has been reduced by approximately 22% at 1mgEP/L and 20% at 10mgEP/mL.

### Example 11

### Enzyme Performance in Treatment of Green Coffee Beans

Green Robusta coffee beans (grown in Vietnam) were incubated at 85 degrees Celsius in two liter per kilogram pre-heated deionized water with 0 or 0.03 or 0.06 or 0.2 or 0.34 or 0.6 milligram per kilogram (enzyme protein weight/weight beans) asparaginase for one hour. The supernatant was removed; and the beans were washed in four liter per kilogram 100 millimolar hydrochloric acid to inactivate the asparaginase, the solution was decanted and the beans were kept in a household sieve to allow the execs liquid to run off. Afterwards the beans were ground in a household coffee grinder. An aliquot of the wet ground green coffee bean powder (2.5 gram) was put into 50 milliliter screw cap tubes and 25 milliliter of 100 millimolar hydrochloric acid was added. The 50 milliliter screw cap tubes were closed and turned end over end for one hour. The hydrochloric acid was filtered through nylon filter with a pore size on 0.22 micrometer. Sample were stored frozen until analysis for dissolved asparagine as described above.

TABLE 9

| Pyrococcus furiosus | |
|---|---|
| % asparagine in beans | mg EP/kg beans |
| 100 | 0 |
| 63 | 0.06 |
| 53 | 0.34 |
| 47 | 0.6 |

TABLE 10

| Thermococcus kodakarensis | |
|---|---|
| % asparagine in beans | mg EP/kg beans |
| 100 | 0 |
| 87 | 0.03 |
| 59 | 0.2 |

1. A method for producing a heat-treated food product comprising:
(a) contacting of a food material with an asparaginase which (i) has at least 60% sequence identity to the mature polypeptide of SEQ ID NO: 10, (ii) is encoded by a polynucleotide having at least 60% sequence identity to SEQ ID NO: 9, or (iii) is a variant of the mature polypeptide of SEQ ID NO: 10 comprising a substitution, deletion, and/or insertion at one or more positions; and
(b) heat-treating the asparaginase treated food material to obtain the heat-treated food product.

2. The method of claim 1, wherein the asparaginase has sequence identity to the mature polypeptide of SEQ ID NO: 10.

3. The method of claim 1, wherein the asparaginase is a thermostable asparaginase.

4. The method of claim 1, wherein in step (a), the asparaginase is added to the food material at a temperature of at least 60° C., such as at least 80° C.

5. The method of claim 1, wherein the contacting in step (a) is for at least 2 minutes.

6. The method of claim 1, wherein the asparaginase is obtained from Thermococcus, preferably from Thermococcus kodakaraensis.

7. The method of claim 1, wherein the heat-treated food product is French fries, and wherein the food material is blanched potato strips.

8. The method of claim 1, wherein the heat-treated food product is sliced potato chips, and wherein the food material is potato slices.

9. The method of claim 1, wherein the heat-treated food product is a potato-based food product and wherein the food material to be treated with asparaginase is mashed potato, a potato-based dough or a suspension of a dehydrated potato product, such as potato flakes or granules.

10. The method of claim 1, wherein the heat-treated food product is a breakfast cereal and wherein the food material comprises whole or processed cereal kernels or grains.

11. The method of claim 1, wherein the heat-treated food product is roasted coffee beans and wherein the food material is unroasted coffee beans or a water extract of unroasted coffee beans.

12. An isolated polynucleotide having at least 60% sequence identity to SEQ ID NO: 9, which encodes an asparaginase.

13. A nucleic acid construct or expression vector comprising the polynucleotide of claim 12 operably linked to one or more control sequences that direct the production of the asparaginase in a Bacillus strain.

14. A recombinant Bacillus host cell comprising the polynucleotide of claim 12 operably linked to one or more control sequences that direct the production of the asparaginase.

15. A method of producing an asparaginase, comprising:
(a) cultivating the Bacillus host cell of claim 14 under conditions conducive for production of the asparaginase; and
(b) recovering the asparaginase.


SEQUENCE LISTING (10 sequences)

SEQ ID NO: 1 - DNA, 987 bp, Thermococcus kodakaraensis
  CDS (1)..(987)
atgaaacttctggttctcggcacggggggcaccatagcgagtgcaaagacggagatgggatacaaggcagcgctcagtgcagatgatatccttcagctggcgggcattaggcgagaagacggtgccaagatagaaacccgggacatcctgaaccttgacagcactctcatacagccagaggattgggtgactatcggacgggcggtttttgaggcttttgatgaatacgacggtatagtaataacacacggaacagacacactcgcctacacttcttccgcccttagcttcatgataaggaatccgccgatacccgttgttctgaccggttcaatgctcccgataaccgagccaaacagcgacgcacccagaaaccttagaacggcactcacctttgcaaggaaaggatttcccgggatatacgtggctttcatggacaagataatgctcgggacgcgcgtttctaaggttcactccctcggcctcaacgcctttcagagtatcaattacccagacatagcctacgtgaagggtgacgaagtcctcgttagacataagcccagaattgggaatggtgaaccattgtttgatccagagcttgaccctaacgttgtccatatcagacttactccagggctttcaccggaggttcttagggcagtcgccagggccacagatggaatcgtcctcgagggttacggagcaggtggtatcccatacaggggcagaaatctcctggaggtagtctctgagacagcgagagaaaaaccggttgtcatgacgacgcaggctctctacgggggagttgatctgacgcgctatgaagttgggagaagggcccttgaagccggcgtgatcccagcgggcgacatgacaaaggaagcgactttgacaaagctcatgtgggcgctgggacacaccagagatcttgaagaaatcagaaaaattatggaacgaaacatcgcgggcgaaatcactgggagttaa

SEQ ID NO: 2 - protein, 328 aa, Thermococcus kodakaraensis
MKLLVLGTGGTIASAKTEMGYKAALSADDILQLAGIRREDGAKIETRDILNLDSTLIQPEDWVTIGRAVFEAFDEYDGIVITHGTDTLAYTSSALSFMIRNPPIPVVLTGSMLPITEPNSDAPRNLRTALTFARKGFPGIYVAFMDKIMLGTRVSKVHSLGLNAFQSINYPDIAYVKGDEVLVRHKPRIGNGEPLFDPELDPNVVHIRLTPGLSPEVLRAVARATDGIVLEGYGAGGIPYRGRNLLEVVSETAREKPVVMTTQALYGGVDLTRYEVGRRALEAGVIPAGDMTKEATLTKLMWALGHTRDLEEIRKIMERNIAGEITGS

SEQ ID NO: 3 - protein, 328 aa, Thermococcus sp. AM4
MRILIMGTGGTIASAKTERGYKAKLSVDEILAIAGIRGDGVKIETRDVLNVDSTLMQPEDWITIGEAVFESLNDYDGIVITHGTDTLAYTSSALSFMLRNVPIPVVLTGSMLPITEPNSDAPRNLKTALTFAMKGFPGIYVAFMDKIMLGTRVSKVHSLGLNAFQSINYPDIAYIKGDEIIVRHKPELPVGEPSFDPRIDPNVVHIRLTPGLSPEVFLAVAERVHGIVLEGYGAGGIPYRGRNLLEAVSKVAREKPVVMTTQALYGGVDLTRYEVGRRALEAGVIPAGDMTKEATLVKLMYALGRTREIEEVRRIMEKNLAGELSSAF

SEQ ID NO: 4 - protein, 328 aa, Thermococcus gammatolerans
MRILIIGTGGTIASSKTERGYKATLSVDEILEIAGIRGDGVKIDTKDVLNIDSTLIQPEDWITIGKAVFESLNDYDGIVITHGTDTLAYTSSALSFMLRNVPIPIVLTGSMLPITEPNSDAPRNLKTALTFAMKGFSGIYVAFMDKIMLGTRVSKVHSLGLNAFQSINYPDIAYIKGEEVVIRHRPELPTGEPSFDPRIDPNVAYLRLTPGLSPEVFLAVAEKVHGIVLEGYGAGGIPYRGRNLLEAVSRVAREKPVVMTTQALYGGVDLRRYEVGRRALEAGVIPAGDMTKEATLVKLMYALGRTNGLEEVRRIMEKNLAGELSSAF

SEQ ID NO: 5 - protein, 331 aa, Thermococcus sibiricus
MKKLLIIGTGGTIASAKTEQGYKSVLKIDEILKLAKIKLENGYKIDSTNIMNIDSTLIHPEDWEIIAKEVFKALDDYDGIIITHGTDTLAYTASMLSFMIKNPNKPIVLTGSMLPITENGSDAPRNIRTAIKFAMEDVAGVFVAFMDKIMLGCRTSKVRTLGLNAFMSINYPDVAYVKGEKILYNIPKEKFQPNGSPELDTKYEPRVVVLRVTPGLGGEIIDAVLDAGYKGIVLEGYGAGGLPYRKSNLLSKIKEITPKIPVIMTTQALYDGVDMRKYEVGRKALETGIIPAKDMTKEATITKLMWALGHTKDVEKIREIMHTNYVNEIKS

SEQ ID NO: 6 - protein, 326 aa, Pyrococcus furiosus
MKILLIGMGGTIASVKGENGYEASLSVKEVLDIAGIKDCEDCDFLDLKNVDSTLIQPEDWVDLAETLYKNVKKYDGIIVTHGTDTLAYTSSMISFMLRNPPIPIVFTGSMIPATEENSDAPLNLQTAIKFATSGIRGVYVAFNGKVMLGVRTSKVRTMSRDAFESINYPIIAELRGEDLVVNFIPKFNNGEVTLDLRHDPKVLVIKLIPGLSGDIFRAAVELGYRGIVIEGYGAGGIPYRGSDLLQTIEELSKEIPIVMTTQAMYDGVDLTRYKVGRLALRAGVIPAGDMTKEATVTKLMWILGHTNNVEEIKVLMRKNLVGELRD

SEQ ID NO: 7 - DNA, 38 bp, Artificial Sequence
  Fwd oligomer
aaaggagaggataaagaatgaaacttcttgttcttggt

SEQ ID NO: 8 - DNA, 43 bp, Artificial Sequence
  Reverse oligomer
gcgtttttttattgattaacgcgtagaacctgtgatttcacca

SEQ ID NO: 9 - DNA, 987 bp, Artificial Sequence
  Synthetic gene
atgaaactgctggttctgggcacaggcggaacaattgcatcagcaaaaacagaaatgggctataaagcagcactgtcagcagatgatattctgcaactggcaggcattagaagagaagatggcgcaaaaattgaaacacgcgatattctgaacctggattcaacactgattcaaccggaagattgggttacaattggcagagcagtttttgaagcgtttgatgaatatgatggcatcgttattacgcatggcacagatacactggcatatacatcatcagcactgtcatttatgattcgcaatccgcctattccggttgttctgacaggctcaatgctgccgattacagaaccgaattcagatgcaccgagaaatctgagaacagcactgacatttgcgagaaaaggctttccgggaatttatgttgcgtttatggataaaatcatgctgggcacaagagttagcaaagttcattcactgggcctgaatgcatttcagtcaattaactatccggatatcgcgtatgtcaaaggcgacgaagttctggttagacataaaccgagaattggcaatggcgaaccgctgtttgatccggaactggatccgaatgttgttcatattcgcctgacaccgggactgtcaccggaagttctgagagcagttgcaagagcgacagatggcattgttctggaaggctatggcgcaggcggaattccgtatagaggcagaaatctgctggaagttgtttcagaaacagcaagagaaaaaccggttgtgatgacaacacaagcactgtatggcggagttgatctgacaagatatgaagttggcagacgcgcactggaagcaggcgttattccggcaggcgatatgacaaaagaagcaacactgacaaaactgatgtgggcactgggccatacaagagatctggaagaaattcgcaaaatcatggaacgcaatattgcaggcgaaattacaggctcataa

SEQ ID NO: 10 - protein, 328 aa, Artificial Sequence; identical to SEQ ID NO: 2
  Synthetic Construct